(19) United States
(12) Patent Application Publication
(10) Pub. No.: US 2023/0010500 A1
(43) Pub. Date: Jan. 12, 2023

(54) SWITCHED CAPACITOR-BASED ELECTRICAL STIMULATION DEVICE AND METHOD

(71) Applicant: Korea University Research and Business Foundation, Seoul (KR)

(72) Inventors: Hyung-Min LEE, Seoul (KR); Kyeongho EOM, Seoul (KR); Han-Sol LEE, Seoul (KR); Minju PARK, Seoul (KR)

(73) Assignee: Korea University Research and Business Foundation, Seoul (KR)

(21) Appl. No.: 17/825,605

(22) Filed: May 26, 2022

(30) Foreign Application Priority Data
Jul. 6, 2021 (KR) 10-2021-0088377

(51) Int. Cl. A61N 1/36; A61N 1/378; A61N 1/04; H02J 7/34

(52) U.S. Cl. CPC A61N 1/36014; A61N 1/36125; A61N 1/378; A61N 1/0492; H02J 7/345

(57) ABSTRACT

Provided is a switched capacitor-based electrical stimulation device which supplies a direct current (DC) power, detects a charging voltage charged in any one of a plurality of capacitors, controls the DC power supplied to a capacitor module to repeat a charging level and a resting level according to a charging pattern when the charging voltage is lower than a target voltage, and outputs an electric current to electrodes which contact a human body based on an output pattern.

### CROSS-REFERENCE TO RELATED APPLICATION

This application claims priority to Korean Patent Application No. 10-2021-0088377, filed on Jul. 6, 2021, and all the benefits accruing therefrom under 35 U.S.C. § 119, the contents of which in its entirety are herein incorporated by reference.

### BACKGROUND

### 1. Field

The present disclosure relates to a switched capacitor-based electrical stimulation device and method, and more particularly, to an electrical stimulation device and method for applying electrical stimulation by switching a plurality of capacitors.

### 2. Description of the Related Art

Implantable medical devices implanted in the human brain or organs to measure neural signals and apply stimulation are important technology for neuroscience research and disease treatment. The implantable medical devices achieve ultrasmall design, low power and high performance in combination with integrated circuit technology. The implantable medical devices include wireless power transfer, neural signal measurement and electrical stimulators. Among them, the electrical stimulators consume a lot of power and may cause damage to cell tissues when they malfunction, so prudent design is required.

The electrical stimulators stimulate nerves by transmitting electric charges to the tissues in the human body through electrodes. The implantable neural electrical stimulators are used in cardiac pacemakers, cochlear implants, bladder stimulators, neuromuscular stimulators, deep brain stimulators, bionic eyes and the like. The electrical stimulators having low power efficiency generate a large amount of heat, causing damage to cells, and have shorter battery run time due to high energy consumption. Additionally, to implant in the human body, the electrical stimulators need to be small in size and safe to prevent electric charges from being continuously accumulated in cells. Even with electrical stimulation of equal energy, it is necessary to select the optimal stimulation waveform to obtain high treatment efficacy. To stimulate multiple sites, multichannel stimulators are preferred. High current intensity, high frequency and high pulse width modulator resolution are required.

The commonly used electric stimulators generate a constant voltage from a direct current (DC)-DC converter, operate a constant current source from the voltage and supply an electric current to the electrodes. However, accumulation of electric charges in the electrodes and the tissues may cause damage to the electrodes and the tissues.

### SUMMARY

The present disclosure is directed to providing a switched capacitor-based electrical stimulation device and method for applying electrical stimulation to the human body by charging a plurality of capacitors with voltage and sequentially discharging the capacitors through electrodes attached to the human body.

An aspect of the present disclosure may include a power module to supply a direct current (DC) power; a capacitor module including a plurality of capacitors which is charged with the DC power; a charging module to detect a charging voltage charged in any one of the plurality of capacitors, and control the DC power supplied to the capacitor module to repeat a charging level and a resting level according to a preset charging pattern when the charging voltage is lower than a preset target voltage; and an output module including electrodes which contact a human body, to receive the power charged in the capacitor and output an electric current to the electrodes based on a preset output pattern.

Additionally, the charging module may generate a rising voltage which rises according to a preset slope from a preset initial voltage to the target voltage, and charge the capacitor module at a time when the rising voltage and the charging voltage are equal.

Additionally, when the charging module charges the capacitor module, the charging module may control to sequentially increase a time interval of the charging level from a minimum time interval to a maximum time interval within a preset time cycle.

Additionally, the output module may receive the power from an arbitrary first capacitor among the plurality of capacitors, output the electric current to the electrodes according to a preset first pattern, receive the power from a second capacitor that is different from the first capacitor among the plurality of capacitors when the first pattern ends, output the electric current to the electrodes according to a second pattern of different polarity from the first pattern, and ground the electrodes when the second pattern ends.

Additionally, the charging module may include a voltage source which outputs a preset maximum target voltage, a plurality of switches connected at one side to the voltage source, a resistor which connect opposite sides of the different adjacent switches, and a resistor which grounds an opposite side of the switch positioned at a lowest end, to set the target voltage corresponding to a set voltage inputted from a user by turning on the switch corresponding to the set voltage.

Another aspect of the present disclosure provides an electrical stimulation method in a switched capacitor-based electrical stimulation device, including supplying, by a power module, a DC power; charging a plurality of capacitors included in a capacitor module with the DC power; detecting, by a charging module, a charging voltage charged in any one of the plurality of capacitors, and controlling the DC power supplied to the capacitor module to repeat a charging level and a resting level according to a preset charging pattern when the charging voltage is lower than a preset target voltage; and receiving, by an output module including electrodes which contact a human body, the power charged in the capacitor, and outputting an electric current to the electrodes based on a preset output pattern.

Additionally, the charging module may generate a rising voltage which rises according to a preset slope from a preset initial voltage to the target voltage, and charge the capacitor module at a time when the rising voltage and the charging voltage are equal.

Additionally, when the charging module charges the capacitor module, the charging module may control to sequentially increase a time interval of the charging level from a minimum time interval to a maximum time interval within a preset time cycle.

Additionally, the output module may receive the power from an arbitrary first capacitor among the plurality of capacitors, output the electric current to the electrodes according to a preset first pattern, receive the power from a second capacitor that is different from the first capacitor among the plurality of capacitors when the first pattern ends, output the electric current to the electrodes according to a second pattern of different polarity from the first pattern, and ground the electrodes when the second pattern ends.

Additionally, the charging module may include a voltage source which outputs a preset maximum target voltage, a plurality of switches connected at one side to the voltage source, a resistor which connects opposite sides of the different adjacent switches and a resistor which grounds an opposite side of the switch positioned at a lowest end, to set the target voltage corresponding to a set voltage inputted from a user by turning on the switch corresponding to the set voltage.

According to an aspect of the present disclosure, the switched capacitor-based electrical stimulation device and method may apply electrical stimulation to the human body by charging the plurality of capacitors with voltage and sequentially discharging the capacitors through the electrodes attached to the human body.

### BRIEF DESCRIPTION OF THE DRAWINGS

FIG. 1 is a schematic diagram of a switched capacitor-based electrical stimulation device according to an embodiment of the present disclosure.

FIG. 2 is a circuit diagram showing an embodiment of a power module of FIG.

FIG. 3 is a circuit diagram showing an embodiment of a capacitor module of FIG. 1.

FIGS. 4 to 6 are circuit diagrams showing an embodiment of a charging module of FIG. 1.

FIG. 7 is a circuit diagram showing an embodiment of an output module of FIG. 1.

FIG. 8 is a circuit diagram showing an embodiment of the electrical stimulation device of FIG. 1.

FIG. 9 is a flowchart of an electrical stimulation method according to an embodiment of the present disclosure.

### DETAILED DESCRIPTION

The following detailed description of the present disclosure is made with reference to the accompanying drawings, in which particular embodiments for practicing the present disclosure are shown for illustrative purposes. These embodiments are described in sufficiently detail for those skilled in the art to practice the present disclosure. It should be understood that various embodiments of the present disclosure are different but do not need to be mutually exclusive. For example, particular shapes, structures and features described herein in connection with one embodiment may be implemented in other embodiment without departing from the spirit and scope of the present disclosure. It should be further understood that changes may be made to the positions or placement of individual elements in each disclosed embodiment without departing from the spirit and scope of the present disclosure. Accordingly, the following detailed description is not intended to be taken in limiting senses, and the scope of the present disclosure, if appropriately described, is only defined by the appended claims along with the full scope of equivalents to which such claims are entitled. In the drawings, similar reference signs denote same or similar functions in many aspects.

Hereinafter, preferred embodiments of the present disclosure will be described in more detail with reference to the accompanying drawings.

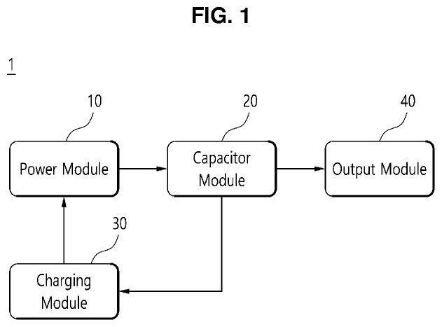

FIG. 1 is a schematic diagram of a switched capacitor-based electrical stimulation device according to an embodiment of the present disclosure.

The switched capacitor-based electrical stimulation device 1 (hereinafter, electrical stimulation device) may determine a target voltage of a charging voltage charged in a capacitor module 20 according to a set voltage inputted from a user.

Here, the set voltage may be understood as a digital input selected from the user, and accordingly, the electrical stimulation device 1 may generate the target voltage corresponding to the set voltage, and through this, the electrical stimulation device 1 may be provided to cause the charging voltage charged in the capacitor module 20 to reach the target voltage.

In this instance, the charging voltage may refer to the magnitude of voltage that is charged in the capacitor module 20, and the target voltage may refer to the magnitude of voltage that will be charged in the capacitor module 20. Additionally, the capacitor module 20 may include a plurality of capacitors.

Accordingly, the electrical stimulation device 1 may include electrodes that contact the human body to output the voltage charged in the capacitor through the electrodes, and through this, the electrical stimulation device 1 may apply electrical stimulation to the human body.

Here, the human body may refer to human skin as well as human brain or organs, and in this case, the electrical stimulation device 1 may be implanted in the human body to receive external power through wireless power transfer technology, and additionally, the electrical stimulation device 1 may receive the settings of the electrical stimulation to be applied to the human body inputted from the user through wireless power transfer technology.

To this end, the electrical stimulation device 1 may include a power module 10, the capacitor module 20, a charging module 30 and an output module 40.

Additionally, the electrical stimulation device 1 may be implemented by a smaller or larger number of components than those shown in FIG. 1. Alternatively, the electrical stimulation device 1 may include at least two components combined into a single component which performs the combined functions. Hereinafter, the above-described components will be described.

The power module 10 may supply direct current (DC) power. In this instance, the power module 10 may be provided to supply the DC power from a battery, and the power module 10 may be provided to wirelessly induce external power.

In this case, the power module 10 may include a LC oscillator to induce the external power, and in this instance, the LC oscillator may be provided to induce the power with a change in external magnetic fields.

Accordingly, the power module 10 may convert the induced power to DC voltage using an alternating current (AC)-DC rectifier. Through this, the power module 10 may supply the converted DC power.

In this instance, the power module 10 may include a plurality of transistors 13, 15 to control the direction of transfer of the DC power.

Here, the transistors 13, 15 may be a Bipolar Junction Transistor (BJT) device in which an electric current flows between a collector and an emitter based on voltage applied between a base and the emitter. Alternatively, the transistors 13, 15 may be a Metal-Oxide-Semiconductor Field-Effect Transistor (MOSFET) device in which an electric current flows between a drain and a source based on voltage applied between a gate and the source.

Accordingly, the power module 10 may be provided to control the magnitude of the DC power according to the voltage applied to the transistors 13, 15, and the power module 10 may be provided to control the direction of the DC power according to the voltage applied to the transistors 13, 15.

To this end, the power module 10 may include a power unit 11, a first transistor 13, a second transistor 15 and an inductor 17.

Accordingly, the power unit 11 may generate the DC power, and the first transistor 13 may be connected to one side of the power unit 11 and the inductor 17 to interrupt or conduct the DC power that is transmitted to the capacitor module 20.

Additionally, the second transistor 15 may be connected to ground one side of the inductor 17 to control the level of the DC power that is transmitted to the capacitor module 20.

In this instance, the level of the DC power may include a charging level and a resting level, and in this instance, the charging level may refer to the level of the DC power generated by the power unit 11, and the resting level may refer to the level of the ground by the second transistor 15.

For example, the charging level may refer to a high level of DC power of a square wave shape, and the resting level may refer to a low level of DC power of a square wave shape.

The inductor 17 may connect the capacitor module 20 to the first transistor 13, and accordingly, the inductor 17 may transmit the DC power received from the power unit 11 to the capacitor module 20.

The capacitor module 20 may include a plurality of capacitors that is charged with the DC power.

The charging module 30 may detect the charging voltage charged in any one of the plurality of capacitors, and when the charging voltage is lower than the preset target voltage, the charging module 30 may control the DC power supplied to the capacitor module 20 to repeat the charging level and the resting level according to a preset charging pattern.

Here, the preset charging pattern may refer to a pattern in which the time interval of the charging level sequentially increases from the minimum time interval to the maximum time interval within an arbitrary time cycle.

In this case, the charging pattern may be understood as a pattern in which the time interval of the resting level sequentially decreases from the maximum time interval to the minimum time interval within the same time cycle.

Accordingly, when the charging module 30 charges the capacitor module 20, the charging module 30 may perform control to sequentially increase the time interval of the charging level from the minimum time interval to the maximum time interval within a preset time cycle.

For example, the charging module 30 may perform Pulse Width Modulation (PWM) by controlling the first transistor 13 and the second transistor 15 of the power module 10, and in this case, the charging module 30 may control the DC power transmitted from the power module 10 to the capacitor module 20 in a sequential order from the lowest duty of PWM to the highest duty of PWM.

Through this, the charging module 30 may gradually charge the capacitor module 20, and in other words, the charging module 30 may gradually increase the charging voltage of the capacitor module 20.

In relation to this, the charging module 30 may generate a rising voltage that rises according to a preset slope from a preset initial voltage to the target voltage, and the charging module 30 may charge the capacitor module 20 at the time when the rising voltage and the charging voltage are equal.

Here, the preset initial voltage may be set to a voltage of the capacitor module 20 in fully discharged state, and the preset slope may be set to the same slope as a charging voltage vs time graph during the charge of the capacitor module 20.

Through this, the charging module 30 may detect the charging voltage remaining in any one of the plurality of capacitors after the output from the output module 40, and the charging module 30 may charge the corresponding capacitor from the remaining charging voltage to the target voltage.

In this instance, the charging module 30 may be provided to detect the charging voltage of a capacitor that is different from the capacitor used to output the electric current from the output module 40.

Meanwhile, the charging module 30 may set a plurality of target voltages corresponding to the set voltage inputted from the user. Through this, the charging module 30 may charge the capacitor module 20 based on the target voltage corresponding to the set voltage.

To this end, the charging module 30 may include a voltage source that outputs a preset maximum target voltage, the charging module 30 may include a plurality of switches connected at one side to the voltage source, and the charging module 30 may include a resistor that connects the opposite sides of the different adjacent switches and a resistor that grounds the opposite side of the switch positioned at the lowest end.

Accordingly, the charging module 30 may be provided to set the target voltage corresponding to the set voltage by turning on the switch corresponding to the set voltage inputted from the user.

For example, the charging module 30 may set the target voltage of 1 V, 1.5 V, 2 V, 2.5 V and 3 V, and in this case, the charging module 30 may set the maximum target voltage of 3 V.

The output module 40 may include electrodes that contact the human body, the output module 40 may receive the power charged in the capacitor, and the output module 40 may output the electric current to the electrodes based on a preset output pattern.

Through this, the output module 40 may apply electrical stimulation to the human body by sequentially discharging the plurality of capacitors.

In this instance, the preset output pattern may be set to a combination of the DC power transmitted from the power module 10 and the charging voltage charged in the capacitor module 20.

For example, the output module 40 may be set to interrupt the DC power transmitted from the power module 10 and output the electric current to the electrodes using only the charging voltage charged in the capacitor, and in this case, the output pattern may be similar to a graph of a decreasing exponential function.

Additionally, the output module 40 may be set to interrupt the DC power transmitted from the power module 10 after maintaining the DC power for a preset time interval and output the electric current to the electrodes using only the charging voltage charged in the capacitor, and in this case, the output pattern may be similar to a graph of an exponential function that decreases after it maintains for the corresponding time interval.

Meanwhile, the output module 40 may receive the power from an arbitrary first capacitor among the plurality of capacitors, the output module 40 may output the electric current to the electrodes according to a preset first pattern, the output module 40 may receive the power from a second capacitor that is different from the first capacitor among the plurality of capacitors when the first pattern ends, the output module 40 may output the electric current to the electrodes according to a second pattern of different polarity from the first pattern, and the output module 40 may ground the electrodes when the second pattern ends.

In this instance, the output module 40 may perform control to charge the capacitor corresponding to the corresponding pattern up to the charging voltage immediately before it outputs the electric current to the electrodes according to each pattern.

Meanwhile, the first pattern and the second pattern may be the same output pattern, and in this instance, the first pattern and the second pattern may be only set to have the opposite polarity.

For example, the output module 40 may control an arbitrary output pattern to output the electric current through the positive electrode immediately after the charge of the first capacitor, and in this instance, the output module 40 may charge the second capacitor after the end of the output of the electric current by the first capacitor. Accordingly, the output module 40 may control the same output pattern to output the electric current through the negative electrode immediately after the charge of the second capacitor.

Accordingly, the output module 40 may ground the electrodes after the end of the output of the electric current by the second capacitor. The output module 40 may perform control to remove the electric charge remaining in the electrodes.

In this instance, the output module 40 may set the output of the electric current by the first capacitor, the output of the electric current by the second capacitor and the grounding of the electrodes as an output cycle, and in this case, the output module 40 may repeat the output cycle.

As described above, the electrical stimulation device 1 may detect the remaining voltage of the capacitor and perform charging from the corresponding remaining voltage to the target voltage, and through this, the electrical stimulation device 1 may provide relatively high charging efficiency.

In this instance, the electrical stimulation device 1 may generate electrical stimulation by sequentially charging and discharging the plurality of capacitors, and through this, the electrical stimulation device 1 may achieve efficient power management.

Additionally, the electrical stimulation device 1 may apply electrical stimulation to the human body in the shape of a graph of a decreasing exponential function by discharging the capacitor module 20, and through this, the electrical stimulation device 1 may carry out electrical stimulation of higher efficacy.

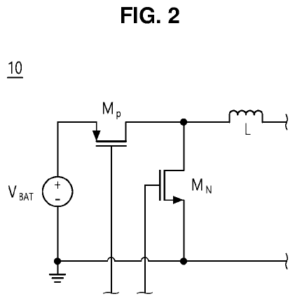

FIG. 2 is a circuit diagram showing an embodiment of the power module of FIG. 1.

Referring to FIG. 2, V_BAT may denote the power unit 11, M_P may denote the first transistor 13, M_N may denote the second transistor 15, and L may denote the inductor 17.

Accordingly, the power unit 11 may generate the DC power, and the first transistor 13 may be connected to one side of the power unit 11 and the inductor 17 to interrupt or conduct the DC power that is transmitted to the capacitor module 20.

Additionally, the second transistor 15 may be connected to ground one side of the inductor 17 to control the level of the DC power that is transmitted to the capacitor module 20.

Additionally, the inductor 17 may connect the capacitor module 20 to the first transistor 13, and accordingly, the inductor 17 may transmit the DC power transmitted from the power unit 11 to the capacitor module 20.

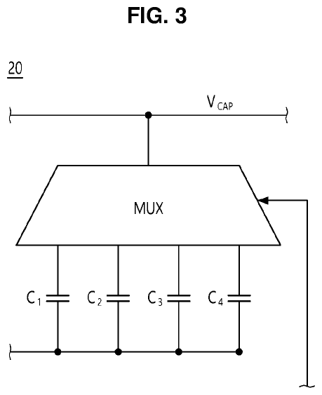

FIG. 3 is a circuit diagram showing an embodiment of the capacitor module of FIG. 1.

Referring to FIGS. 3, C_1 to C_4 may denote the plurality of capacitors, and V_CAP may denote the charging voltage. Additionally, MUX may be provided to connect any one of the plurality of capacitors to the output module 40 or the charging module 30.

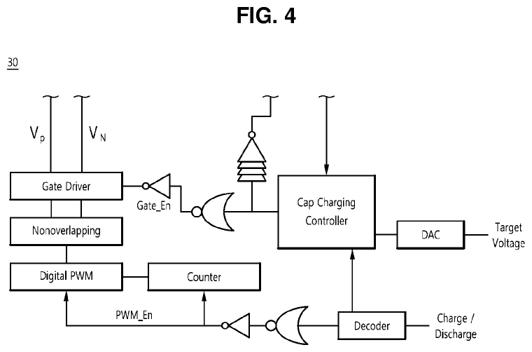

FIGS. 4 to 6 are circuit diagrams showing an embodiment of the charging module of FIG. 1.

Referring to FIG. 4, V_P may be an input signal for the first transistor 13, and V_N may be an input signal for the second transistor 15.

Additionally, Gate Driver may be a module that generates a signal to cause the first transistor 13 and the second transistor 15 to conduct or interrupt the electric current, and Nonoverlapping may be a module that controls Gate Driver to prevent the first transistor 13 and the second transistor 15 from being set to equally conduct or interrupt the electric current. Additionally, Digital PWM may be a module that generates the charging level and the resting level of the power module 10.

In this instance, Counter may be provided to measure the time interval from the time when the charging of the capacitor module 20 is performed, and accordingly, Digital PWM may perform control to sequentially reduce the charging level from the maximum time interval to the minimum time interval at the time interval of Counter.

Additionally, DAC may generate the target voltage corresponding to the set input from the user, and in this instance, Target Voltage inputted to DAC may be the maximum target voltage.

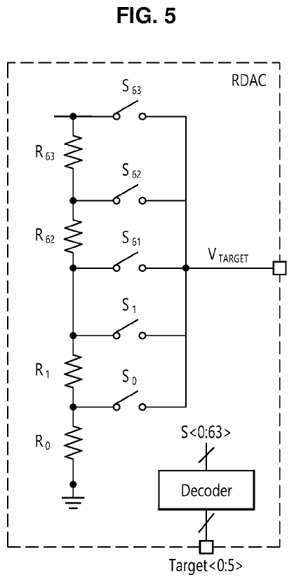

In relation to this, referring to FIG. 5, an embodiment of DAC is shown, and in this instance, DAC used in an embodiment may be a Resistive Digital-to-Analog Converter (RDAC).

Accordingly, RDAC may include a plurality of switches S_0, S_1, . . . , S_63 where V_TARGET is applied to one side, and RDAC may include resistors R_1, R_2, . . . , R_63 that connect the opposite sides of the different adjacent switches and resistor R_0 that grounds the opposite side of the switch S_0 positioned at the lowest end.

Accordingly, RDAC may set the target voltage corresponding to the set voltage by turning on any one switch corresponding to the set voltage inputted from the user.

Meanwhile, in FIG. 4, Cap Charging Controller may refer to a module that detects the charging voltage charged in any one of the plurality of capacitors and determines if the charging voltage is lower than the preset target voltage.

In this instance, Cap Charging Controller may generate a rising voltage that rises according to the preset slope from the preset initial voltage to the target voltage and determine the time when the rising voltage and the charging voltage are equal.

Accordingly, Cap Charging Controller may determine whether to charge the capacitor module 20 based on the charging voltage, the target voltage and the rising voltage.

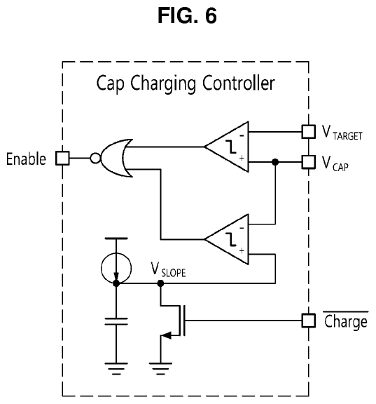

In relation to this, referring to FIG. 6, V_TARGET may denote the target voltage, V_CAP may denote the charging voltage, V_SLOPE may denote the rising voltage, and Charge may denote the state of charge of the capacitor module 20.

Accordingly, when V_SLOPE is smaller than V_CAP and V_TARGET is larger than V_CAP, Cap Charging Controller may perform control to charge the capacitor module 20.

In this instance, when V_SLOPE is larger than V_CAP or V_TARGET is smaller than V_CAP, Cap Charging Controller may perform control to stop charging the capacitor module 20.

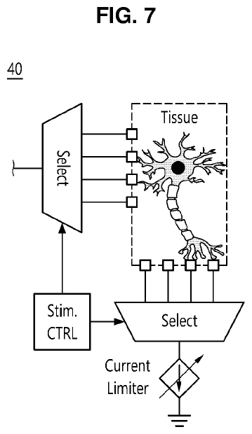

FIG. 7 is a circuit diagram showing an embodiment of the output module of FIG. 1.

Referring to FIG. 7, Select may be a module that selects a channel to which voltage will be applied from the capacitor module 20 among different channels. Here, the channel may refer to an electrode pair that contacts the human body at different locations, and the electrode pair may refer to a pair of the electrode connected to the capacitor module 20 and the grounded electrode.

In this instance, Current Limiter may be provided to limit the amount of electric current outputted to the electrodes, and through this, Current Limiter may protect each module in the electrical stimulation device 1 or prevent the flow of excessive current in the human body.

Additionally, Stim. CTRL may be a module that controls Select or controls the output pattern of the electric current that will be transmitted to the electrodes.

Accordingly, Stim. CTRL may receive the power from an arbitrary first capacitor among the plurality of capacitors, output the electric current to the electrodes according to the preset first pattern, receive the power from the second capacitor that is different from the first capacitor among the plurality of capacitors when the first pattern ends, output the electric current to the electrodes according to the second pattern of different polarity from the first pattern, and connect Select connected to the capacitor module 20 to the grounded Select to ground the electrodes when the second pattern ends.

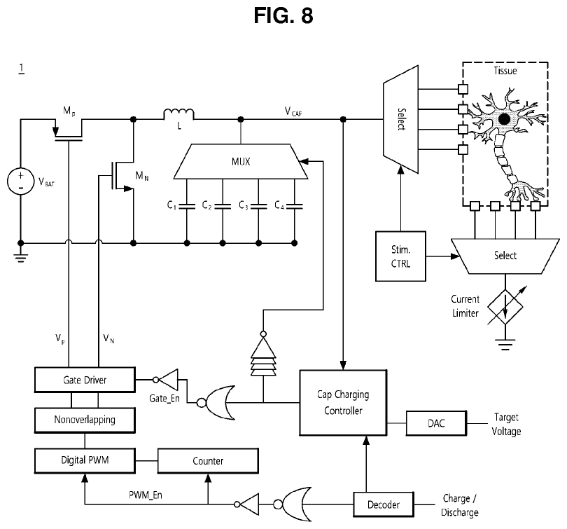

FIG. 8 is a circuit diagram showing an embodiment of the electrical stimulation device of FIG. 1.

Referring to FIG. 8, an interconnect circuit diagram of an embodiment according to FIGS. 2 to 7 is shown.

Through this, the electrical stimulation device 1 may detect the remaining voltage of the capacitor module 20 and perform charging from the corresponding remaining voltage to the target voltage, and through this, the electrical stimulation device 1 may provide relatively high charging efficiency.

Additionally, the electrical stimulation device 1 may apply electrical stimulation to the human body in the shape of a graph of a decreasing exponential function by discharging the capacitor module 20, and through this, the electrical stimulation device 1 may carry out electrical stimulation of higher efficacy.

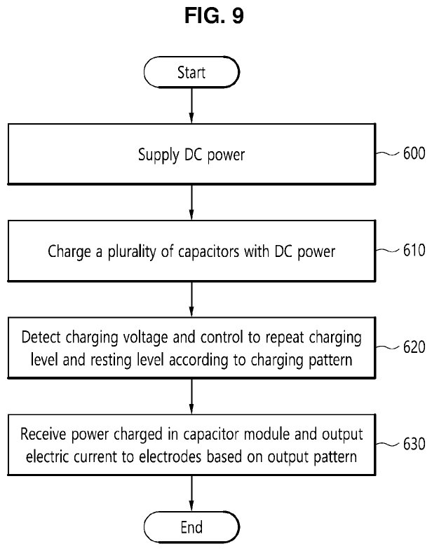

FIG. 9 is a flowchart of an electrical stimulation method according to an embodiment of the present disclosure.

Since the electrical stimulation method according to an embodiment of the present disclosure is performed on substantially the same configuration as the electrical stimulation device 1 shown in FIG. 1, the same component as the electrical stimulation device 1 of FIG. 1 is given the same reference sign, and redundant description is omitted.

The method may include the steps of supplying, by the electrical stimulation device 1, the DC power (600), charging the plurality of capacitors with the DC power (610), detecting the charging voltage and controlling to repeat the charging level and the resting level according to the charging pattern (620), and receiving the power charged in the capacitor module and outputting the electric current to the electrodes based on the output pattern (630).

The step 600 of supplying the DC power may be a step in which the power module 10 supplies the DC power.

The step 610 of charging the plurality of capacitors with the DC power may be a step in which the plurality of capacitors included in the capacitor module 20 is charged with the DC power.

The step 620 of detecting the charging voltage and controlling to repeat the charging level and the resting level according to the charging pattern may be a step in which the charging module 30 detects the charging voltage charged in any one of the plurality of capacitors, and controls the DC power supplied to the capacitor module 20 to repeat the charging level and the resting level according to the preset charging pattern when the charging voltage is lower than the preset target voltage.

The step 630 of receiving the power charged in the capacitor module and outputting the electric current to the electrodes based on the output pattern may be a step in which the output module 40 includes the electrodes that contact the human body, and receives the power charged in the capacitor and outputs the electric current to the electrodes based on the preset output pattern.

While the present disclosure has been hereinabove described with reference to the embodiments, those skilled in the art will understand that a variety of modifications and changes may be made thereto without departing from the spirit and scope of the present disclosure defined in the appended claims.

### DETAILED DESCRIPTION OF MAIN ELEMENTS

1: Electrical stimulation device
10: Power module
20: Capacitor module
30: Charging module
40: Output module

What is claimed is:

1. A switched capacitor-based electrical stimulation device, comprising:
a power module to supply a direct current (DC) power;
a capacitor module including a plurality of capacitors which is charged with the DC power;
a charging module to detect a charging voltage charged in any one of the plurality of capacitors, and control the DC power supplied to the capacitor module to repeat a charging level and a resting level according to a preset charging pattern when the charging voltage is lower than a preset target voltage; and
an output module including electrodes which contact a human body, to receive the power charged in the capacitor and output an electric current to the electrodes based on a preset output pattern.

2. The switched capacitor-based electrical stimulation device according to claim 1, wherein the charging module generates a rising voltage which rises according to a preset slope from a preset initial voltage to the target voltage, and charges the capacitor module at a time when the rising voltage and the charging voltage are equal.

3. The switched capacitor-based electrical stimulation device according to claim 1, wherein when the charging module charges the capacitor module, the charging module controls to sequentially increase a time interval of the charging level from a minimum time interval to a maximum time interval within a preset time cycle.

4. The switched capacitor-based electrical stimulation device according to claim 1, wherein the output module receives the power from an arbitrary first capacitor among the plurality of capacitors, outputs the electric current to the electrodes according to a preset first pattern, receives the power from a second capacitor that is different from the first capacitor among the plurality of capacitors when the first pattern ends, outputs the electric current to the electrodes according to a second pattern of different polarity from the first pattern, and grounds the electrodes when the second pattern ends.

5. The switched capacitor-based electrical stimulation device according to claim 1, wherein the charging module includes a voltage source which outputs a preset maximum target voltage, a plurality of switches connected at one side to the voltage source, a resistor which connect opposite sides of the different adjacent switches, and a resistor which grounds an opposite side of the switch positioned at a lowest end, to set the target voltage corresponding to a set voltage inputted from a user by turning on the switch corresponding to the set voltage.

6. An electrical stimulation method in a switched capacitor-based electrical stimulation device, the electrical stimulation method comprising:
supplying, by a power module, a direct current (DC) power;
charging a plurality of capacitors included in a capacitor module with the DC power;
detecting, by a charging module, a charging voltage charged in any one of the plurality of capacitors, and controlling the DC power supplied to the capacitor module to repeat a charging level and a resting level according to a preset charging pattern when the charging voltage is lower than a preset target voltage; and
receiving, by an output module including electrodes which contact a human body, the power charged in the capacitor, and outputting an electric current to the electrodes based on a preset output pattern.

7. The electrical stimulation method according to claim 6, wherein the charging module generates a rising voltage which rises according to a preset slope from a preset initial voltage to the target voltage, and charges the capacitor module at a time when the rising voltage and the charging voltage are equal.

8. The electrical stimulation method according to claim 6, wherein when the charging module charges the capacitor module, the charging module controls to sequentially increase a time interval of the charging level from a minimum time interval to a maximum time interval within a preset time cycle.

9. The electrical stimulation method according to claim 6, wherein the output module receives the power from an arbitrary first capacitor among the plurality of capacitors, outputs the electric current to the electrodes according to a preset first pattern, receives the power from a second capacitor that is different from the first capacitor among the plurality of capacitors when the first pattern ends, outputs the electric current to the electrodes according to a second pattern of different polarity from the first pattern, and grounds the electrodes when the second pattern ends.

10. The electrical stimulation method according to claim 6, wherein the charging module includes a voltage source which outputs a preset maximum target voltage, a plurality of switches connected at one side to the voltage source, a resistor which connects opposite sides of the different adjacent switches and a resistor which grounds an opposite side of the switch positioned at a lowest end, to set the target voltage corresponding to a set voltage inputted from a user by turning on the switch corresponding to the set voltage.
